(19) United States
(12) Patent Application Publication
(10) Pub. No.: US 2026/0216718 A1
(43) Pub. Date: Jul. 30, 2026

(54) MEDIUM, DEVICE AND METHOD FOR MONITORING REPORTING AND VERIFICATION OF CARBON DIOXIDE REMOVAL

(71) Applicant: Mati Carbon India Private Limited, Delhi (IN)

(72) Inventors: Robin Marlar Rajendran, Delhi (IN); Anikendra Das, Delhi (IN); Jacob S. Jordan, Houston, TX (US); Shantanu Agarwal, Houston, TX (US)

(21) Appl. No.: 19/463,305

(22) Filed: Jan. 29, 2026

(30) Foreign Application Priority Data
Jan. 29, 2025 (IN) 202511007369

(51) Int. Cl. B01J 41/05; B01J 41/07; B01J 41/14; B01J 47/018; G01N 33/24

(52) U.S. Cl. CPC B01J 41/05; B01J 41/07; B01J 41/14; B01J 47/018; G01N 33/24

(57) ABSTRACT

The invention pertains to a novel medium, device and method for monitoring, reporting, and verification (MRV) of CDR through ERW in agricultural soils. The device is based on anion-exchange resin and employs a medium comprising defined mass ratio of anion-exchange resin and rigid, inert, non-porous material to quantify bicarbonate flux in terrestrial soil. The device of the present invention continuously adsorbs bicarbonate over the cropping season, thereby providing a time-integrated measure of total carbon removal rather than a single point-in-time measurement. The method of the present invention involves among other aspects, strategic placement of the device near a field bund enabling accurate representation of field-level carbon flux.

### FIELD OF THE INVENTION

The present invention is in the field of carbon dioxide removal (CDR) technologies. More specifically, the invention pertains to a novel medium, device and method for monitoring, reporting, and verification (MRV) of carbon dioxide removal in enhanced rock weathering.

### BACKGROUND OF THE INVENTION

As the global community seeks effective solutions to combat climate change, negative emissions technologies (NETs) have emerged as critical tools for removing carbon dioxide (CO₂) from the atmosphere. Among these, enhanced rock weathering (ERW) has been identified as a potentially scalable and cost-effective carbon dioxide removal (CDR) approach with associated environmental and agronomic benefits. ERW involves the application of finely crushed silicate minerals to agricultural soils to accelerate natural weathering reactions. These reactions capture atmospheric CO₂, converting it into stable bicarbonate and carbonate ions that are stored in soils or transported to aquatic systems, where they can remain sequestered for millennia. While the potential of ERW is clear, its practical deployment at scale is hindered by the lack of reliable and efficient methods for monitoring, reporting, and verifying (MRV) the amount of CO₂ removed.

Existing MRV methods aim to track carbon removal by measuring specific indicators. For example, some soil-based approaches track changes in the cation inventory of the solid phase. Concentrations of base cations (e.g., Na, K, Mg, Ca) can then be used to infer the amount of CO₂ produced and transported out of the system. Despite the high analytical precision of these measurements, their indirect linkage to CO₂ removal limits their effectiveness for MRV applications.

A common MRV approach involves analysing dissolved inorganic carbon (DIC), particularly bicarbonate ions, in soil and water. Techniques like coulometry and spectroscopy are highly accurate but require specialized equipment and expertise, making them cost-intensive and impractical for widespread use in large agricultural fields (Amann et al., 2022; Almaraz et al., 2022). Soil based-mass-balance methodologies involving Inductively Coupled Plasma-Mass Spectrometry (ICP-MS) measurements help practitioners track CO₂ removal by monitoring the solid residue (Reershemius et al., 2023), but these methods have proven time consuming and costly. Another method involves monitoring the accumulation of exchangeable ions in soils using ion-exchange resins. While this approach has been explored in patents, such as U.S. Pat. No. 11,644,454, and has been applied in studies for measuring nutrients like nitrate and ammonia (Kantola et al., 2023), its use for directly quantifying bicarbonate ions in enhanced weathering processes remains underdeveloped.

Zero-Ex has developed a resin package for the capture of the dissolved molecules produced by weathering which utilizes mixed bed resins. WO2025064488 discloses an alkalinity sensor which uses a cation exchange resin intended for MRV of ERW.

The primary limitations of current MRV techniques include high operational costs, methodological complexity, and uncertainties associated with adapting these methods to diverse environmental conditions. These constraints not only increase the financial burden of implementing ERW but also make it difficult to provide accurate and timely data to validate carbon removal claims.

Further, most existing MRV approaches rely on complex geochemical models or grab samples of soil, and are often inaccurate due to the high variability of open-field environments. Traditional high-accuracy methods (such as sophisticated sensors, ICPMS, etc.) are expensive and difficult to scale across thousands of hectares of agricultural land. Methods presently used often provide “snapshots” of data that fail to capture the continuous flux of carbon sequestration over an entire growing season. Methods that focus on tracking carbonate precipitation in soils are valuable for assessing long-term storage but fall short in providing real-time data through bicarbonate capture and are influenced by site-specific variations in soil chemistry. In addition, without direct, physical evidence of bicarbonate capture, carbon registries are hesitant to issue high-value credits, slowing down the financial viability of ERW projects.

Such bottlenecks create a pressing need for an innovative, scalable, and cost-effective MRV system that can deliver precise measurements while addressing these challenges.

### SUMMARY OF THE INVENTION

It is therefore a general object of the present invention to advance monitoring, reporting and verification of carbon dioxide removal in soil. In particular, it is an object of the present invention to provide a medium, device and method for MRV of CDR that are cost-effective and scalable. It is also an object of the present invention to provide a medium, device and method that enable accurate determination of CDR.

The general object of the present invention is achieved through a scalable, low-cost medium, device and method that enable MRV of CDR through capture and quantification of bicarbonate ions generated by silicate weathering reactions in the ERW process. In particular, the present invention employs a medium comprising an anion exchange resin mixed with a rigid, inert, non-porous material. Such a medium that comprises an anion exchange resin mixed with a rigid, inert, non-porous material in a pre-determined ratio, has a porosity similar to that of soil and enables water percolation, ion transport and reactive surface accessibility similar to that of soil thereby enabling more accurate quantification of soil carbon dioxide flux. Thus, the medium, device and method of the present invention are not only low-cost and scalable but also provide a more accurate representation of carbon flux by mimicking the surrounding soil's flow conditions. The device is deployable in situ and is capable of continuously accumulating bicarbonate ions from soil infiltrate over time while buried in the soil. This enables the generation of a time-integrated, cumulative measure of CO₂ sequestration, rather than a single point-in-time estimate, thereby improving the accuracy, reliability, and verifiability of ERW-related carbon dioxide removal measurements. Similarly, the method of the present invention which employs the novel device ensures cost-effective, scalable and accurate data collection. Additionally, the method of the present invention may employ a combination of the novel device with the defined strategy of placing copies of the device in an array near the bunds in a field to achieve better and more accurate data collection.

Thus, in an aspect the present invention provides a medium for adsorbing and storing bicarbonate ions from a sample, such as soil, wherein the medium comprises an anion exchange resin and a rigid, inert, non-porous material, wherein the mass ratio of the anion exchange resin to the rigid, inert, non-porous material is from about 1:5 to 1:10.

In some embodiments, the present invention provides a medium for adsorbing and storing bicarbonate ions from a sample, such as soil, wherein the medium comprises a mixture of an anion exchange resin and beads of a rigid, inert, non-porous material, wherein the mass ratio of the anion exchange resin to beads of the rigid, inert, non-porous material is from about 1:5 to 1:10.

In some embodiments, the mass ratio of the anion exchange resin to rigid, inert, non-porous material is from about 1:5 to 1:6. In some embodiments, the anion exchange resin is selected from the group consisting of a strong-base anion exchange resin, a weak-base anion exchange resin, a gel-type anion exchange resin and combinations thereof. In some embodiments, the anion exchange resin is polystyrene-based anion exchange resin or acrylic polymer-based anion exchange resin. In some embodiments, the anion exchange resin is in hydroxide (OH⁻) form or chloride (Cl⁻) form or in both hydroxide (OH⁻) and chloride (Cl⁻) forms. In some embodiments, the anion exchange resin is operational over a pH range of 0 to 14. In some embodiments, the rigid, inert, non-porous material is selected from the group consisting of glass, quartz, ceramic and combinations thereof. In some embodiments, the rigid, inert, non-porous material is glass. In some embodiments, the rigid, inert, non-porous material is in the form of beads. In some embodiments, the rigid, inert, non-porous material is glass beads. In some embodiments, the beads of the rigid, inert, non-porous material have an average diameter between about 0.6 mm and 1 mm.

In another aspect, the present invention provides a device for monitoring, reporting and verification of carbon dioxide removal in enhanced rock weathering, the device comprising three chambers, the chambers being a first chamber, an intermediate chamber and a second chamber. The intermediate chamber is present between the first chamber and the second chamber. The first chamber and the second chamber house soil and the intermediate chamber houses a medium for adsorbing and storing bicarbonate ions. The first chamber and the second chamber are separated from the intermediate chamber by porous layers and the porous layers are configured to prevent the movement of soil from the first chamber and second chamber into the intermediate chamber while allowing the soil infiltrate to pass through the three chambers of the device. The medium for adsorbing and storing bicarbonate ions present in the intermediate chamber comprises an anion exchange resin and a rigid, inert, non-porous material in a pre-determined mass ratio.

In an embodiment, the present invention provides a device (1) for monitoring, reporting and verification of carbon dioxide removal in enhanced rock weathering, the device (1) comprising three chambers (2, 3, 4), the chambers being a first chamber (2), an intermediate chamber (3) and a second chamber (4). The intermediate chamber (3) is present between the first chamber (2) and the second chamber (4). The first chamber (2) and the second chamber (4) house soil and the intermediate chamber (3) houses a medium (7) for adsorbing and storing bicarbonate ions. The first chamber (2) and the second chamber (4) are separated from the intermediate chamber (3) by porous layers (5, 6). The porous layers (5, 6) are configured to prevent the movement of soil from the first chamber (2) and second chamber (4) into the intermediate chamber (3) while allowing the soil infiltrate to pass through the chambers (2, 3, 4) of device (1). The medium (7) for adsorbing and storing bicarbonate ions present in the intermediate chamber (3) comprises an anion exchange resin and a rigid, inert, non-porous material in a pre-determined mass ratio.

In some embodiments of the device, the anion exchange resin and rigid, inert, non-porous material are present in a mass ratio from about 1:5 to 1:10. In some embodiments of the device, the anion exchange resin and rigid, inert, non-porous material are present in a mass ratio from about 1:5 to 1:6. In some embodiments of the device, the rigid, inert, non-porous material is in the form of particles or beads. In some embodiments of the device, the rigid, inert, non-porous material is in the form of beads. In some embodiments, the beads have an average diameter between about 0.6 mm and 1 mm. In some embodiments of the device, the rigid, inert, non-porous material is selected from the group consisting of glass, quartz, ceramic and combinations thereof. In some embodiments of the device, the rigid, inert, non-porous material is glass, preferably glass beads. In some embodiments, the glass beads have an average diameter between about 0.6 mm and 1 mm.

In some embodiments of the device, the anion exchange resin is selected from the group consisting of a strong-base anion exchange resin, a weak-base anion exchange resin, a gel-type anion exchange resin and combinations thereof. In some embodiments of the device, the anion exchange resin is polystyrene-based anion exchange resin or acrylic polymer-based anion exchange resin. In some embodiments of the device, the anion exchange resin is in hydroxide (OH⁻) form or chloride (Cl⁻) form or in both hydroxide (OH⁻) and chloride (Cl⁻) forms. In some embodiments of the device, the anion exchange resin is operational over a pH range of 0 to 14. In some embodiments of the device, the porous layer is a mesh or a porous substrate. In some embodiments of the device, the porous layers are a nylon mesh, a filtration cloth, a microporous synthetic mesh, or combinations thereof.

In some embodiments, the first and second chambers (2, 4) of the device (1) get filled with soil when the device (1) is deployed in the field. In other embodiments of the invention, the first chamber (2) and the second chamber (4) are filled with soil prior to deploying them in the field.

In some embodiments, the device comprises a housing (8) which is composed of a material that is impermeable, water-resistant and capable of maintaining structural integrity. In some embodiments, the device comprises a housing (8) which is composed of a material selected from the group consisting of polyvinyl chloride (PVC), polypropylene, high density polyethylene, and is preferably polyvinyl chloride.

In some embodiments, the device (1) is designed to comply with equation (1)

$$K\_avg = {L}_{1} / \{ ( {L}_{2} / {K}_{2} ) + ( {L}_{3} / {K}_{3} ) + ( {L}_{4} / {K}_{4} ) \} \qquad ( 1 )$$

wherein,
K_avg is the average permeability of the device in m/s;
L₁ is the total length of the device (1),
L₂, L₃ and L₄ are the lengths of the first (2), intermediate (3) and second chambers (4), respectively, and
K₂, K₃ and K₄ are the permeability in m/s of the first (2), intermediate (3) and second (4) chambers, respectively.

In another embodiment, the device could be a pre-assembled device or could be a device assembled in situ, that is, assembled at the field site where the device has to be deployed.

In another aspect, the present invention provides the use of a device (1) of invention for monitoring, reporting and verification of carbon dioxide removal in enhanced rock weathering.

In another aspect, the present invention relates to a method for monitoring, reporting and verification of carbon dioxide removal in enhanced rock weathering, comprising:
a. positioning one or more device (1) in soil such that an upstream opening (9) of the device (1) is arranged underneath a soil surface (11) in a field,
b. allowing water present in the field to pass through the device (1);
c. retrieving the device (1) from the soil after a pre-determined period of time; and
d. quantifying the bicarbonate ions taken up by the medium (7) for adsorbing and storing bicarbonate ions.

In some embodiments of the method, the device could be a pre-assembled device or could be a device assembled in situ, that is, assembled at the field site where the device has to be deployed.

In some embodiments of the method the pre-determined period of time is at least one cropping season. In some embodiments of the method the quantification of bicarbonate ions is carried out by elution and titration. In some embodiments of the method, the device (1) is positioned vertically in the soil. In some embodiments of the method, the device (1) is positioned near the bund in a field. In some embodiments of the method, the multiple devices (1) are positioned near the bund in a field at a repeated and regular interval. In some embodiments of the method, the multiple devices (1) are positioned along the bund in a field at a repeated and regular interval of 2 meters from each other.

In another aspect, the present invention provides a use of a method of the invention for monitoring, reporting and verification of carbon dioxide removal in enhanced rock weathering.

### BRIEF DESCRIPTION OF THE DRAWINGS

The following drawings are illustrative of particular examples for enabling the device and methods of the present disclosure, which are descriptive of some of the devices, methods and mechanism and are not intended to limit the scope of the invention. The drawings are not to scale (unless so stated) and are intended for use in conjunction with the explanations in the following detailed description.

FIG. 1 exemplarily illustrates an isometric or wire-frame view of an embodiment of the device for monitoring, reporting and verification of carbon dioxide removal in enhanced rock weathering. The device (1) comprises three chambers—a first chamber (2), an intermediate chamber (3) and a second chamber (4). The first chamber (2) and second chamber (4) are separated from the intermediate chamber by porous layers (5, 6).

FIG. 2 exemplarily illustrates an isometric view of the device (1) arranged underneath a soil surface (11).

FIG. 3 exemplarily illustrates a top view of a field where multiple copies of the device of the present invention are arranged at regular intervals along the bund (12) in a field. The point of insertion of the device is represented by 13.

FIG. 4 illustrates calibration curves obtained in a laboratory experiment conducted to evaluate the adsorption of bicarbonate ions by various ion-exchange resins under no flow conditions. The calibration curves were obtained by plotting the amount of bicarbonate ions (mg) adsorbed by the resin against solute (NaHCO₃) concentration of the feed. In each experiment mass of resin used was 1 g and elution was carried out with 100 ml of 1M NaCl. FIG. 4(a) shows the calibration curve obtained for strong base type 1 anion exchange resin, FIG. 4(b) shows the calibration curve obtained for strong base type 2 anion exchange resin, FIG. 4(c) shows the calibration curve obtained for weak-base anion exchange resin, and FIG. 4(d) shows the calibration curve obtained for gel type anion exchange resin.

FIG. 5 illustrates the calibration curves obtained in a laboratory experiment conducted to evaluate the adsorption of bicarbonate ions by various ion-exchange resins under flow conditions. The calibration curves were obtained by plotting the adsorbed bicarbonate ions (mg) against solute (NaHCO₃) concentration of the feed. In each experiment the feed flow rate was maintained at 100 mL/h, the mass of resin used was 1 g and elution was carried out with 100 ml of 1M NaCl. FIG. 5(a) shows the calibration curve obtained for strong base type 1 anion exchange resin, FIG. 5(b) shows the calibration curve obtained for strong base type 2 anion exchange resin, FIG. 5(c) shows the calibration curve obtained for weak-base anion exchange resin, and FIG. 5(d) shows the calibration curve obtained for gel type anion exchange resin.

FIG. 6 illustrates the average bicarbonate amount adsorbed by devices of the present invention comprising different types of anion exchange resins (strong base type 1 anion exchange and gel type anion exchange) when used under field conditions. The devices were retrieved 120 days after their deployment in fields, the bicarbonate ions were eluted from the anion exchange resin (strong base type 1 anion exchange or gel type anion exchange) and then titrated to obtain the amount of bicarbonate ions adsorbed to the resin. The control bars demonstrate results of experiments which were run in fields with soil that was not treated with silicates for enhanced rock weathering while the experimental bars demonstrate results of experiments which were run in fields with soil that was treated with silicates for enhanced rock weathering. The devices used in the control and experimental runs for each anion exchange resin type were identical in design and medium composition.

Persons skilled in the art will appreciate that elements in the drawings are illustrated for simplicity and clarity and may represent both hardware and software components of the system. Further, the dimensions of some of the elements in the drawings may be exaggerated relative to other elements to help to improve understanding of various exemplary embodiments of the present disclosure. Throughout the drawings, it should be noted that like reference numbers are used to depict the same or similar elements, features, and structures.

### DETAILED DESCRIPTION OF THE INVENTION

Exemplary embodiments will now be described. The disclosure may, however, be embodied in many different forms and should not be construed as limited to the embodiments set forth herein; rather, these embodiments are provided so that this disclosure will be thorough and complete, and will fully convey its scope to those skilled in the art. The terminology used in the detailed description of the particular exemplary embodiments illustrated in the accompanying drawings is not intended to be limiting. In the drawings, like numbers refer to like elements.

It is to be noted, however, that the reference numerals used herein illustrate only typical embodiments of the present subject matter, and are therefore, not to be considered for limiting of its scope, for the subject matter may admit to other equally effective embodiments.

The specification may refer to “an”, “one” or “some” embodiment(s) in several locations. This does not necessarily imply that each such reference is to the same embodiment(s), or that the feature only applies to a single embodiment. Single features of different embodiments may also be combined to provide other embodiments. As used herein, the singular forms “a”, “an” and “the” are intended to include the plural forms as well, unless expressly stated otherwise. It will be further understood that the terms “include”, “comprises”, “including” and/or “comprising” when used in this specification, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this disclosure pertains. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Commercially available ion-exchange resins are conventionally employed for water treatment. These resins are well known to exchange ions but are typically used in controlled flow systems or batch reactors, rather than in heterogeneous soil environments. In heterogeneous soil environments, a device that is filled with resin alone cannot work reliably under real field conditions due to changes in behavior of resin arising from irrigation/rain water contact and from dry spells in land, such as swelling/shrinkage, changes in permeability of the resin, etc. Thus, when resins are deployed alone in soil, this behavior leads to swelling/shrinkage, collapse of pore connectivity, and unpredictable permeability, resulting in diffusion-limited flow and highly variable bicarbonate capture. Overall, this makes resin-only devices unsuitable for the time-integrated bicarbonate measurement of ERW.

The inventors of the present application modified existing resins by mixing the ion-exchange resin with a rigid, inert, non-porous material, creating a mechanically stable and hydraulically controlled medium that can operate under in situ soil conditions without altering permeability due to resin swelling or shrinkage. The mixing of the rigid, inert, non-porous material with the resin helps to stabilize the porosity of the medium and achieves soil-equivalent permeability for an entire deployment period. Thus, the present invention provides an improved medium and device that is specifically adapted for flooded and heterogeneous agricultural environments.

A further improvement offered by the present invention is through the field deployment configuration of the device, wherein the device comprising resin mixed with a rigid, inert, non-porous material is buried at targeted locations within fields. These targeted locations are for example, areas near the bunds in a field. As used herein, the term “bunds” includes bunds of fields, and perimeter areas of a field. As used herein, the term “near the bund in a field” also includes areas around and along the bunds in a field. Other than deploying the device strategically at specific locations, the methods of the present invention also contemplate deployment of multiple devices in spatially distributed networks.

Altogether, the methods and devices of the present invention, alone as well as in combination, enable time-integrated capture of bicarbonate transported through soil water or soil infiltrate.

The present invention relates to the measurement of carbon sequestration via enhanced rock weathering (ERW) using ion-exchange resins to quantify bicarbonate fluxes in terrestrial soils, thereby enabling estimation of ERW efficiency. Rather than relying on mathematical estimations of carbon removal, the invention employs anion-exchange resins to physically capture bicarbonate ions generated during weathering processes. This approach shifts measurement, reporting, and verification from a theoretical estimation to a directly measurable physical outcome. The medium and device of the present invention directly adsorb and store the bicarbonate ions from soil infiltrate. Soil infiltrate is the water that enters or moves through the soil or fills the pores of the soil and typically includes dissolved ions and solutes.

Further, the specific design of the device and the resin-to-rigid, inert, non-porous material ratio present in the medium of the present invention allow the device to function efficiently by relying on media porosity which is very similar to that of soil. The rigid, inert, non-porous material acts as a filler to reduce the effective pore space of the resin which allows the device to accumulate bicarbonate ions all throughout the time that the device remains deployed in the soil. Additionally, the simple design of the device and inclusion of basic elements in the device, such as a housing which could be made of commonly available cheap materials, such as polyvinylchloride, porous layers which could be simple material such as nylon mesh and the use of ion exchange resin and cheap and commonly available rigid, inert, non-porous material such as glass beads in the medium, account for the low-cost of the device.

The simplicity of the device ensures ease of deployment and retrieval, making it suitable for diverse agricultural settings. The device can be buried directly in the soil of agricultural fields treated with silicate minerals. The lack of complex components reduces costs while maintaining the accuracy and reliability of the measurements. The device of the present invention can capture and store carbon signals continuously while buried in the soil and can provide the overall bicarbonate sequestration over time rather than as a single point-in-time reading.

Moreover, the device of the present invention can either be pre-assembled or could even be assembled directly in the field where it has to be deployed with relative ease. To assemble the device in situ, for example, a PVC pipe is taken and a nylon mesh is affixed near a downstream opening of the pipe such that the nylon mesh is placed at a distance from the downstream opening. A mixture of anion exchange resin and glass beads in a pre-determined ratio is then added into the PVC pipe and a second nylon mesh is inserted near the upstream opening of the PVC pipe such that the second nylon mesh is placed at a distance from the upstream opening. This creates three chambers within the PVC pipe—a chamber housing the mixture of anion exchange resin and beads and one chamber each on either side of the chamber housing the mixture of anion exchange resin and beads. In one embodiment, the chambers on either side of the chamber housing the mixture of anion exchange resin and beads can be filled with soil collected from the deployment site and then buried into the deployment site. In alternate embodiments, the chambers on either side of the chamber housing the mixture of anion exchange resin and beads can get filled with soil as the device is buried into the ground at the deployment site. The GPS location of the device is recorded upon installation at the site.

Additionally, the method of monitoring CDR of the present invention which uses the device of the present invention provides several advantages including the ease of handling of device. Further, embodiments of the method where multiple devices of the present invention are deployed at each agricultural site, ensure robust statistical analyses and overcome uncertainties in measurements. After the completion of the crop cycle, the devices are excavated, and the resins are recovered for analysis. Bicarbonate ions adsorbed onto the resins are eluted using a relevant eluate, and their concentrations are quantified using acid-base titration. This process provides a direct measure of the carbon dioxide sequestered during the weathering process. The full bicarbonate flux is estimated using the invention, which informs calculations for carbon dioxide removal.

Further, by burying the device near bunds, embodiments of the method of the present invention target areas where water and solute transport are most representative of the bicarbonate flux of the field. This ensures that the data captured is an accurate reflection of the field's carbon flux, reducing the number of sensors needed and lowering costs. By utilizing standard elution and titration techniques on a novel device, the invention simplifies the laboratory phase. This allows project developers to process thousands of samples using existing infrastructure, making the system highly scalable for climate targets. The “direct-evidence” nature of the resin-captured bicarbonate meets the rigorous standards of carbon registries (such as Isometric or Puro.earth), facilitating the issuance of high-quality carbon removal certificates.

Thus, while existing MRV for ERW typically relies on “mass-balance” accounting and complex geochemical models to estimate how much carbon has been removed the medium, device and method of the present invention provide direct bicarbonate quantification by physically capturing the bicarbonate ions. Further, standard environmental monitoring often uses “grab sampling” (single point-in-time soil or water tests), which can miss the fluctuating carbon pulses caused by rain or seasonal changes. However, the resin-to-rigid, inert, non-porous material mass ratio in the medium and device of the present invention is an unusual feature that allows the device to act as a bicarbonate accumulator. It continuously adsorbs bicarbonate throughout the cropping season, providing a time-integrated total of carbon removal rather than a single snapshot.

Conventional sensors are often placed randomly or in the centre of fields, which may not capture the true “flux” of carbon as it moves out of the soil profile where the sensors are placed. The method of the present invention which deploys the devices in linear arrays along bunds (field boundaries/paddy boundaries) is a distinct procedural improvement. This targets the upstream and downstream soil infiltrate flow paths where bicarbonate transport is most concentrated and ensures that the data is highly representative of the entire field's carbon sequestration performance.

In summary, the medium, devices and methods of the present invention provide the following advantages:
Direct measurement of carbon dioxide removal through direct capture and measurement of bicarbonate ions from soil infiltrate.
Cumulative measurement of bicarbonate ions over the entire time period that the device is deployed in the field.
Data which is most representative of the field is obtained.
Scalability.
Cost effectiveness.

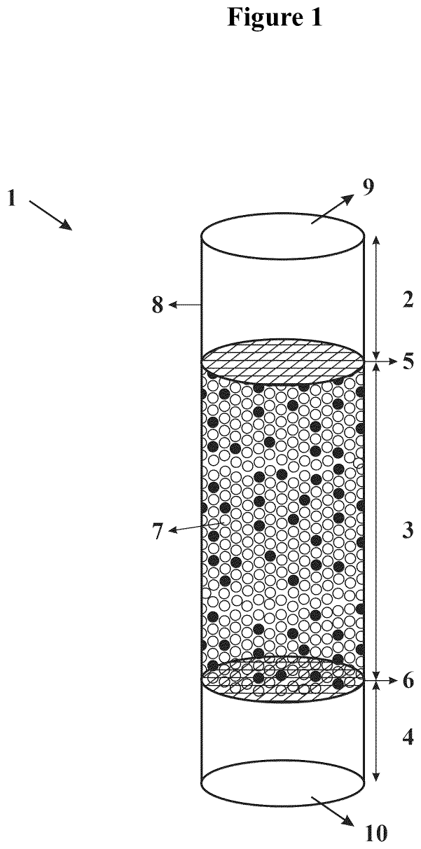

Referring to FIG. 1, FIG. 1 exemplarily illustrates an isometric or wire-frame view of an embodiment of the device for monitoring, reporting and verification of carbon dioxide removal in enhanced rock weathering. The device (1) comprises three chambers—a first chamber (2), an intermediate chamber (3) and a second chamber (4). The first chamber (2) and second chamber (4) are separated from the intermediate chamber by porous layers (5, 6). The first chamber (2) and the second chamber (4) house soil and the intermediate chamber (3) houses a medium (7) for adsorbing and storing bicarbonate ions. The porous layers (5, 6) are configured to prevent the movement of soil from the first chamber (2) and second chamber (4) into the intermediate chamber (3) while allowing the soil infiltrate to pass through the chambers (2, 3, 4) of device (1). The medium (7) for adsorbing and storing bicarbonate ions present in the intermediate chamber (3) comprises an anion exchange resin and a rigid, inert, non-porous material in a pre-determined mass ratio.

The device (3) comprises an upstream opening (9) and a downstream opening (10). The two openings of the device (9, 10) are in direct communication with the soil in which the device is deployed. In some embodiments of the invention, the first chamber (2) and the second chamber (4) get filled with soil when the device is deployed in the soil in the field. In other embodiments of the invention, the first chamber (2) and the second chamber (4) are filled with soil prior to deploying them in the field.

The housing (8) of the device is impermeable, water-resistant and capable of maintaining structural integrity. It could be composed of any material which is impermeable, water-resistant and capable of maintaining structural integrity and can withstand agricultural field conditions, including but not limited to soil, water and microbial activity, without leaching chemicals or plant growth interfering chemicals into the soil. Thus, the housing could be composed of material such as polyvinyl chloride, polypropylene and high-density polyethylene. In preferred embodiments, the housing is composed of polyvinyl chloride. In further preferred embodiments, the housing is a PVC pipe.

The porous layers separating the first and second chambers from the intermediate chamber are a mesh or porous substrate. The ‘mesh or porous substrate’ is any filter material which is chemically inert, mechanically stable and has pore size such that the filter material can retain soil and allow soil infiltrate to pass through it. Thus, the porous layers could be a nylon mesh, a filtration cloth, a microporous synthetic mesh, or combinations thereof. Where a filtration cloth is used as the porous layers, it could be, for example, a 200-mesh size filtration cloth.

The medium present in the intermediate chamber is a novel medium and a novel aspect according to the present invention. The word “medium” as used in the present invention indicates a mixture of an anion exchange resin and a rigid, inert, non-porous material in a pre-determined mass ratio. The pre-determined mass ratio of the anion exchange resin and the rigid, inert, non-porous material is preferably from about 1:5 to 1:10 and more preferably from about 1:5 to 1:6. The novel medium of the present invention which is also present in the intermediate chamber of the device of the present invention, comprises an anion exchange resin and a rigid, inert, non-porous material. The rigid, inert, non-porous material could be in the form of beads or particles. The rigid, inert, non-porous material could be any chemically inert and non-porous material having a controlled particle size and shape and could be for example, glass, ceramic, quartz or combinations thereof. The rigid, inert, non-porous material could be for example, glass beads or ceramic beads or quartz particles. In preferred embodiments, the rigid, inert, non-porous material could be glass beads. In embodiments of the device, rigid, glass beads provide a lattice so that the permeability of the device can remain consistent and predictable. Resins swell and shrink as wetting and drying fronts pass through the device so maintaining a largely constant permeability could be difficult. However, rigid, inert, non-porous material such as glass beads help to overcome this problem and help to form a composite porous medium with controlled and predictable hydraulic behaviour.

The selection of a particular rigid, inert, non-porous material depends on chemical inertness, particle size, sphericity, mechanical strength, and compatibility with the target fluid and resin. Size of the rigid, inert, non-porous material, such as glass beads, is also an important factor that could affect the performance of the medium and device in capturing bicarbonate ions. Beads larger than approximately 1.0 mm may create excessive voids, causing water to channel too quickly through the device for effective ion exchange. Conversely, beads significantly smaller than the resin (e.g., ~0.1 mm) may cause clogging at particle interfaces, disrupting flow and reducing bicarbonate capture efficiency. Thus, for efficient functioning, the beads of the rigid, inert, non-porous material could have an average diameter between 0.6 mm and 1 mm and preferably have an average diameter of 0.8 mm.

The anion exchange resin could be any conventionally used anion exchange resin. For example, the anion exchange resin could be selected from the group consisting of a strong-base anion exchange resin, a weak-base anion exchange resin, a gel-type anion exchange resin and combinations thereof. The anion exchange resin could be a polystyrene-based anion exchange resin or acrylic polymer-based anion exchange resin. The anion exchange resin could be in the hydroxide (OH⁻) form or chloride (Cl⁻) form or in both hydroxide (OH⁻) and chloride (Cl⁻) forms. The anion exchange resin could be operational over a pH range of 0 to 14.

The anion exchange resin is uniformly mixed with the rigid, inert, non-porous material in the medium and device of the present invention.

The device (1) of the present invention can be a pre-assembled device. In alternate embodiments, the device (1) could also be assembled in situ, that is, at the site of deployment in the field. To assemble the device in situ, for example, a PVC pipe is taken and a nylon mesh is affixed near a downstream opening of the pipe such that the nylon mesh is placed at a distance from the downstream opening. A mixture of anion exchange resin and glass beads in a pre-determined ratio is then added into the PVC pipe and a second nylon mesh is inserted near the upstream opening of the PVC pipe such that the second nylon mesh is placed at a distance from the upstream opening. This creates three chambers within the PVC pipe—a chamber housing the mixture of anion exchange resin and beads and one chamber each on either side of the chamber housing the mixture of anion exchange resin and beads. In one embodiment, the chambers on either side of the chamber housing the mixture of anion exchange resin and beads can be filled with soil collected from the deployment site and then buried into the deployment site. In alternate embodiments, the chambers on either side of the chamber housing the mixture of anion exchange resin and beads can get filled with soil as the device is buried into the ground at the deployment site. The GPS location of the device is recorded upon installation at the site.

An important aspect of the present invention is the novel medium which is also used in the novel device of the present invention. During development of the novel medium of the present invention, the inventors found that inclusion of a rigid, inert, non-porous material, such as glass beads, in an anion exchange resin, is essential to create medium porosity similar to that of soil which enables similar water percolation, ion transport, and reactive surface accessibility as that in soil. In the absence of the rigid, inert, non-porous material, such as glass beads, the resin forms a comparatively dense and hydraulically restricted medium which shows reduced effective contact between water and the resin, and non-representative reaction kinetics. Pure anion exchange resin beads, when used alone, tend to pack densely within the cartridge or device, leading to non-uniform pore connectivity and locally restrictive flow that can reduce representative water-resin contact. At the same time, when considered in bulk relative to the surrounding clay soil, a cartridge or device filled only with coarse resin beads would exhibit a much higher permeability than the soil, creating an artificial preferential flow path that could bias the capture of bicarbonate ions by the device.

It is known that the permeability of clay soil is less than 10⁻⁸ m/s. Using the Kozeny-Carman permeability equation, it was seen that pure anion exchange resin (0.315-1.25 mm) in 1 inch PVC pipe when placed in soil creates a high-permeability zone compared to soil. This may affect the exact soil infiltrate flow pattern inside the cartridge and fail to capture a representative signal of carbon sequestration. In order to overcome this issue, the inventors of the present application, uniformly mixed the anion exchange resin with a rigid, inert, non-porous material. It was seen by the inventors of the present application that when a medium comprising the anion exchange resin uniformly mixed with glass beads in a mass ratio of about 1:5 to 1:10, was used in the device, the device could capture a representative flux of carbon by mimicking the surrounding soil's flow conditions. A preferred ratio of anion exchange resin to inert, non-porous material was seen to be in the range of from about 1:5 to about 1:6 (6 g anion exchange resin and 32 g glass beads). It was noted that the rigid, inert, non-porous material acts as a filler to reduce the effective pore space of the device. This tunes the device to behave as an accumulator that mimics the soil, rather than as a drain that attracts unrepresentative water volumes.

The ratio of the anion-exchange resin to the rigid, inert, non-porous material (such as glass beads) directly controls the final porosity of the device and the number of active ion-exchange sites in the device. The device of the present invention has been designed to mimic the real field flow conditions. The device (1) of the present invention complies with the following equation (1)

$$K\_avg = {L}_{1} / \{ ( {L}_{2} / {K}_{2} ) + ( {L}_{3} / {K}_{3} ) + ( {L}_{4} / {K}_{4} ) \} \qquad ( 1 )$$

wherein,
K_avg is the average permeability of the device in m/s;
L₁ is the total length of the device (1),
L₂, L₃ and L₄ are the lengths of the first (2), intermediate (3) and second (4) chambers, respectively, and
K₂, K₃ and K₄ are the permeability in m/s of the first (2), intermediate (3) and second (4) chambers, respectively.

Permeability of the field was taken as the basis for these calculations and the lengths L₂, L₃ and L₄ were iterated accordingly.

Both the device and the medium of the present invention are novel aspects. In an aspect, the present invention discloses a medium for adsorbing and storing bicarbonate ions from a sample and comprises an anion exchange resin and a rigid, inert, non-porous material wherein, the mass ratio of the anion exchange resin to the rigid, inert, non-porous material is from about 1:5 to 1:10 and the medium is soil. Particularly, the medium of the present invention is capable of adsorbing and storing bicarbonate ions from soil infiltrate.

In some embodiments, the mass ratio of the anion exchange resin to the rigid, inert, non-porous material is from about 1:5 to 1:6. In some embodiments, the anion exchange resin is selected from the group consisting of a strong-base anion exchange resin, a weak-base anion exchange resin, a gel-type anion exchange resin and combinations thereof. In some embodiments, the anion exchange resin is polystyrene-based anion exchange resin or acrylic polymer-based anion exchange resin. In some embodiments, the anion exchange resin is in hydroxide (OH⁻) form or chloride (Cl⁻) form or in both hydroxide (OH⁻) and chloride (Cl⁻) forms. In some embodiments, the anion exchange resin is operational over a pH range of 0 to 14. In some embodiments, the rigid, inert, non-porous material is any rigid, inert, non-porous material having controlled particle size and shape and could be selected from the group consisting of glass, quartz, ceramic and combinations thereof. The rigid, inert, non-porous material could be in the form of beads or particles. In some embodiments, the rigid, inert, non-porous material is glass. In some embodiments, the rigid, inert, non-porous material is glass beads. In some embodiments, where the rigid, inert, non-porous material is in the form of beads, the beads of the rigid, inert, non-porous material have an average diameter between about 0.6 mm and 1 mm.

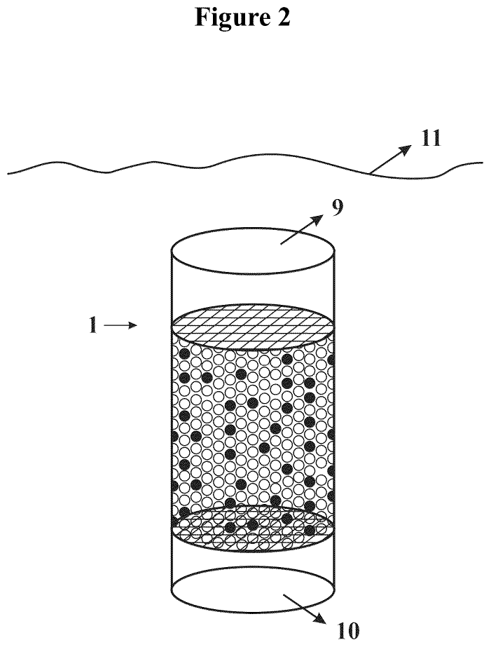

In another novel aspect, the present invention provides a method for monitoring, reporting and verification of carbon dioxide removal in enhanced rock weathering. Referring to FIG. 2, the method comprises positioning one or more device (1) of the present invention in soil such that an upstream opening (9) of the device (1) is arranged underneath a soil surface (11) in a field. After deployment of the device in the field as described above, water present in the field or the soil infiltrate is then allowed to pass through the device (1). Then, the device (1) is retrieved from the soil after a pre-determined period of time and the bicarbonate ions taken up by the medium are quantified.

The pre-determined period of time after which the device is retrieved could be any period of time from one day to a few months. In preferred cases, the device is retrieved after at least one cropping season. The quantification of bicarbonate ions is carried out by elution and titration after the medium is retrieved from the device. In preferred embodiments of the method, the device is positioned vertically in the soil.

The device used in the method can be pre-assembled or could even be assembled directly in the field where it has to be deployed. To assemble the device in situ, for example, a PVC pipe is taken and a nylon mesh is affixed near a downstream opening of the pipe such that the nylon mesh is placed at a distance from the downstream opening. A mixture of anion exchange resin and glass beads in a pre-determined ratio is then added into the PVC pipe and a second nylon mesh is inserted near the upstream opening of the PVC pipe such that the second nylon mesh is placed at a distance from the upstream opening. This creates three chambers within the PVC pipe—a chamber housing the mixture of anion exchange resin and beads and one chamber each on either side of the chamber housing the mixture of anion exchange resin and beads. In one embodiment, the chambers on either side of the chamber housing the mixture of anion exchange resin and beads can be filled with soil collected from the deployment site and then buried into the deployment site. In alternate embodiments, the chambers on either side of the chamber housing the mixture of anion exchange resin and beads can get filled with soil as the device is buried into the ground at the deployment site. The GPS location of the device is recorded upon installation at the site.

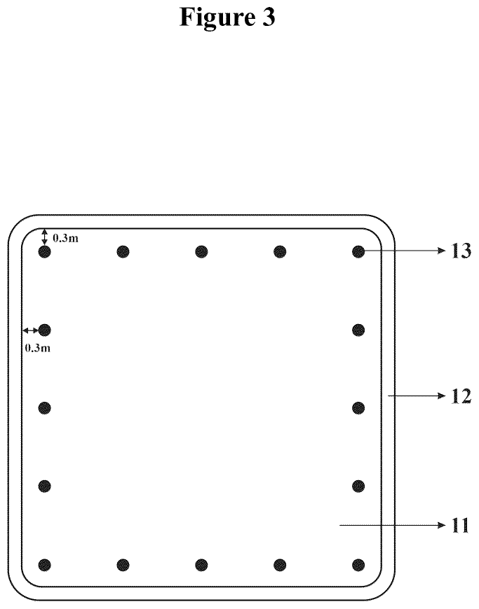

Another important aspect of the method of the present invention is the positioning of the device in the field. Preferably, the device is positioned near the bund in a field as shown in FIG. 3. FIG. 3 illustrates a top view of a field where multiple copies of the device of the present invention are arranged at regular intervals along the bund (12) in a field. The behaviour of water flow in soil is largely governed by the presence of raised earthen boundaries called “bunds” which separate individual fields or individual sections of a field. These bunds are unploughed and less compact than the field interiors. As a result, bunds exhibit significantly higher hydraulic conductivity (Neumann et al., 2009), and large fractions of bicarbonate-rich pore water escape from individual paddies through the bunds (Wopereis et al., 1994; Tuong et al., 1994). Water escaping from an individual field may be transported to adjacent fields or infiltrate downward into deeper groundwater systems. This lateral water movement creates a major blind spot in traditional pore water sampling methodologies, as bicarbonate flux escaping through bunds is rarely captured, leading to severe underestimations of carbon removal when using aqueous-phase MRV approaches in the field. To overcome this drawback and in view of the hydrological flow considerations, the inventors of the present invention deployed the devices near the bund.

As shown in FIG. 3, multiple devices of the present invention could be positioned near the bund (12) in a field at a repeated and regular interval, the point of insertion of the device being represented by (13). In preferred embodiments, multiple devices of the present invention were positioned along the bund in a field at a repeated and regular interval of 2 meters from each other.

The device (1) as used in the method of the present invention can be a pre-assembled device. In alternate embodiments, the device (1) could also be assembled in situ, that is, at the site of deployment in the field. To assemble the device in situ, for example, a PVC pipe is taken and a nylon mesh is affixed near a downstream opening of the pipe such that the nylon mesh is placed at a distance from the downstream opening. A mixture of anion exchange resin and glass beads in a pre-determined ratio is then added into the PVC pipe and a second nylon mesh is inserted near the upstream opening of the PVC pipe such that the second nylon mesh is placed at a distance from the upstream opening. This creates three chambers within the PVC pipe—a chamber housing the mixture of anion exchange resin and beads and one chamber each on either side of the chamber housing the mixture of anion exchange resin and beads. The chambers on either side of the chamber housing the mixture of anion exchange resin and beads can either be filled with soil collected from the deployment site and then buried into the deployment site or can be filled with soil as the device is buried into the ground at the deployment site. The GPS location of the device is recorded upon installation at the site.

The various aspects of the present invention offer several advantages. The device of the present invention functions as an accumulator that continuously records geochemical evidence of carbon sequestration through resin adsorption, soil equivalent permeability, and strategic deployment. Together, the following shows how the invention enables stable, time-integrated, and field-representative bicarbonate capture under field conditions—an outcome that cannot be achieved using resin alone or using conventional soil and water sampling methods.
Ion-exchange capture mechanism: Atmospheric CO₂ consumed during enhanced rock weathering of silicate minerals (e.g., basalt) is converted into bicarbonate ions in soil pore water. The anion-exchange resin within the cartridge is preloaded with mobile counter-ions and has an affinity for bicarbonate. This process immobilizes weathering-derived inorganic carbon in a solid phase that can be recovered and quantified ex situ.
Soil equivalent permeability: An important hypothesis of the invention is that representative bicarbonate capture requires the device to operate within the same hydraulic conditions as the surrounding soil. Resin alone is unsuitable to ensure such operation because swelling, shrinkage, and heterogeneity of the resin create different permeability zones within the device compared to soil. By uniformly mixing the resin with rigid, inert, non-porous material, such as glass beads, at a defined resin-to-rigid, inert, non-porous material mass ratio and preferably using a defined rigid, inert material particle or bead size, a mechanically stable lattice is formed that preserves pore connectivity, maintains permeability similar to the surrounding soil, and ensures that the water and solute flux passing through the device is proportional to the ambient soil flux.
Strategic deployment: The invention further hypothesizes that bicarbonate transport in flooded agricultural systems is spatially organized, with lateral and vertical flow converging toward bunds due to topography, tillage patterns, and water management practices. By deploying resin cartridges near bunds, such as along the bunds of the field, or the bunds of section of the field and in perimeter locations, the system intercepts bicarbonate transported from a broad contributing area of the field, allowing the captured signal to represent integrated field-scale weathering flux rather than isolated point measurements.

The present invention finds application in monitoring, reporting and verification of carbon dioxide removal in enhanced rock weathering, supports research and development on cumulative weathering of feedstock materials, and enables environmental impact assessments of solute transport in agricultural fields and soils. The system provides ERW project developers with a scalable and cost-efficient solution for monitoring carbon sequestration. As a non-intrusive tool, the device can be buried in the field and later recovered without disrupting standard agricultural operations. By providing direct measurements of bicarbonate flux, the invention enhances confidence and transparency for carbon buyers, facilitating reliable carbon accounting and supporting the issuance of high-quality carbon removal certificates. Thus, the medium, devices and methods of the present invention can be effectively used for monitoring, reporting and verification of carbon dioxide removal in enhanced rock weathering.

The examples presented herein are intended to illustrate the invention and are not intended to limit the invention to the specific embodiments disclosed.

### Example 1: Evaluation of Different Anion Exchange Resins for Bicarbonate Adsorption Under No-Flow Conditions and Under Flow Conditions in the Laboratory

### Evaluation of Different Anion Exchange Resins for Bicarbonate Adsorption Under No-Flow Conditions in the Laboratory

Analytical grade NaHCO₃ and H₂SO₄ were used to prepare the standard solution and were diluted further for the experiments. 1.38 g of sodium bicarbonate (NaHCO₃) was weighed and carefully transferred to a clean, dry 1 L volumetric flask. Approximately 500 mL of deionized water was added to the flask and the NaHCO₃ was allowed to dissolve. Once dissolved, deionized water was added to bring the total volume up to 1 liter in the volumetric flask to prepare the stock solution. Similarly, 1 M H₂SO₄ stock solution was also prepared.

Four different types of ion-exchange resins were used in the study: Strong base type 1 anion exchange resin, strong base type 2 anion exchange resin, weak base anion (WBA) exchange resin, and gel-type anion exchange resins. All the resins were procured from Sunresin. A fixed mass of 1 g of each resin type was used for each of the experimental runs. The resins were thoroughly rinsed with deionized water to remove any impurities and to ensure equilibrium prior to testing.

Solutions with varying concentrations of solute (NaHCO₃) were prepared using deionized water. The solute concentrations tested were: 5 mg/L, 10 mg/L, 30 mg/L, 50 mg/L, 80 mg/L, 100 mg/L, 250 mg/L, 500 mg/L, and 1000 mg/L. For each test, the following solution volumes and feed concentrations were used: 20 L for 5 mg/L, 10 L for 10 mg/L, 4 L for 30 mg/L, 2 L for 50 mg/L, 1.5 L for 80 mg/L, 1 L for 100 mg/L, 400 mL for 250 mg/L, 200 mL for 500 mg/L, and 100 mL for 1000 mg/L. Thus, every solution contained at least 100 mg of HCO₃⁻ to ensure sufficient solute-resin interaction. The adsorption of bicarbonate ions (HCO₃⁻) by the four different types of anion-exchange resins was tested under “no flow” conditions. The resins were immersed in their respective solutions and left under no flow conditions for a period of two weeks to allow adsorption.

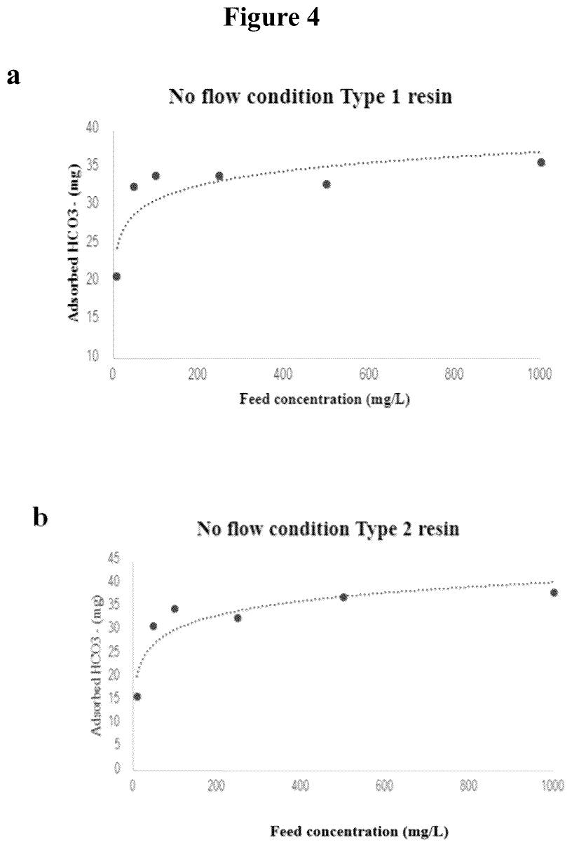

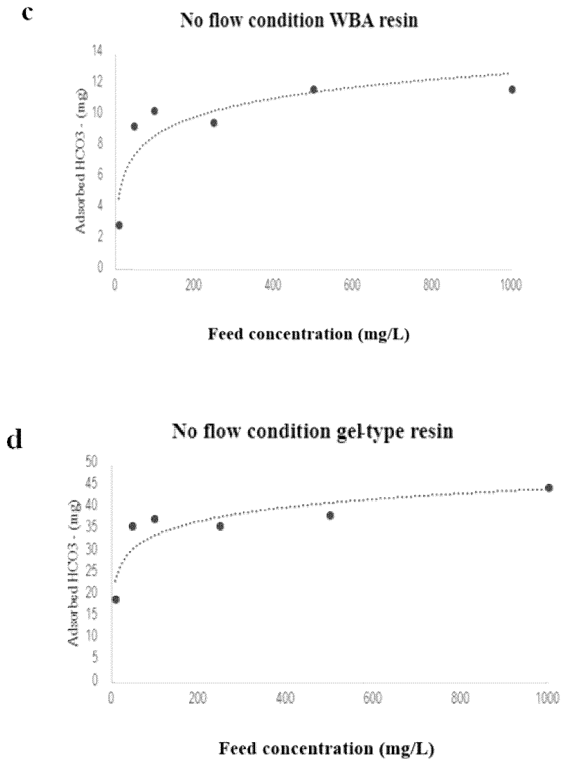

After the two-week period, the bicarbonate ions were eluted from the resins using 100 ml of 1M NaCl to determine the amount of bicarbonate ions adsorbed. The amount of HCO₃⁻ adsorbed was estimated by titration against 0.001 M H₂SO₄. Graphs (FIG. 4) were plotted to show the relationship between the amount of HCO₃⁻ adsorbed by the resins and the feed concentration of the solutions. This graphical representation shows how different feed concentrations influence the adsorption capacity of the tested resins under no flow condition, providing insight into their performance under various contaminant loads.

FIG. 4(a) shows the calibration curve obtained for strong base type 1 anion exchange resin, FIG. 4(b) shows the calibration curve obtained for strong base type 2 anion exchange resin, FIG. 4(c) shows the calibration curve obtained for weak-base anion exchange resin, and FIG. 4(d) shows the calibration curve obtained for gel type anion exchange resin.

### Evaluation of Different Anion Exchange Resins for Bicarbonate Adsorption Under Flow Conditions in the Laboratory

Analytical grade NaHCO₃ and H₂SO₄ were used for preparing the standard solutions which were diluted further for the experiments. 1.38 g of sodium bicarbonate (NaHCO₃) was weighed and carefully transferred to a clean, dry 1 L volumetric flask. Approximately 500 mL of deionized water was added to the flask and the NaHCO₃ was allowed to dissolve. Once the NaHCO₃ was dissolved, deionized water was added to flask to bring the total volume to 1 liter to prepare the stock solution. Similarly, a stock solution of 1M H₂SO₄ was also prepared.

Four different types of ion-exchange resins were used in the study: Strong base type 1 anion exchange, strong base type 2 anion exchange, weak base anion (WBA), and gel-type anion exchange resins. All the resins were procured from Sunresin. A fixed mass of 1 g of each resin type was used for every experimental run. The resins were thoroughly rinsed with deionized water to remove any impurities and to ensure equilibrium prior to testing.

Solutions with varying concentrations of solute (NaHCO₃) were prepared using deionized water. The solute concentrations tested were: 10 mg/L, 50 mg/L, 100 mg/L, 250 mg/L, and 500 mg/L. Experiments were conducted to evaluate the adsorption of bicarbonate ions (HCO₃⁻) by the four different type of anion-exchange resins under “flow” conditions.

A column setup was used to perform the flow condition experiment, with 1 g of resin and a small portion of cotton placed at the bottom of the column to prevent resin loss. A solution of defined concentration was continuously fed into the column, with the flow rate adjusted to 100 mL/h using the column's flow control knob. The column height was kept constant throughout the experiment. Permeate samples were collected every hour, and their bicarbonate concentrations were measured. When the permeate concentration approached the feed concentration, it was assumed the resin had reached saturation. Three consecutive readings were taken to confirm resin saturation.

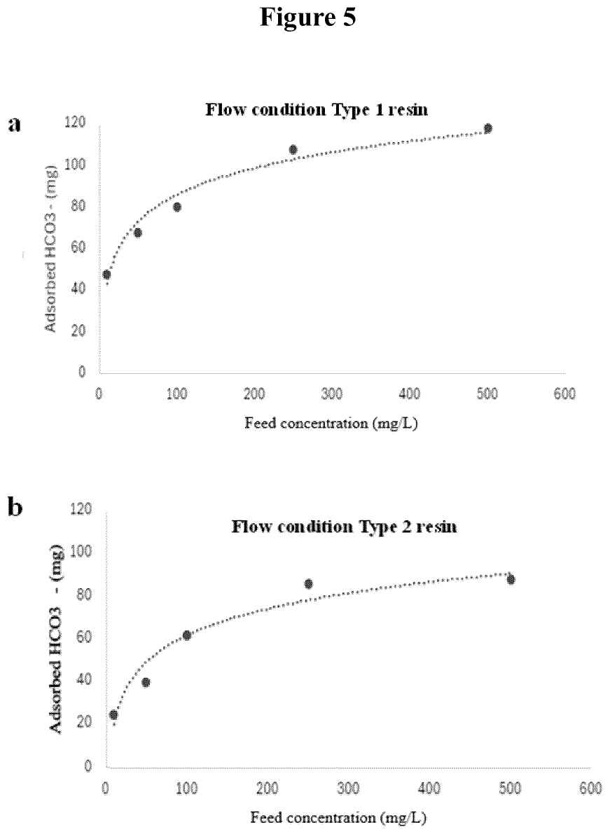

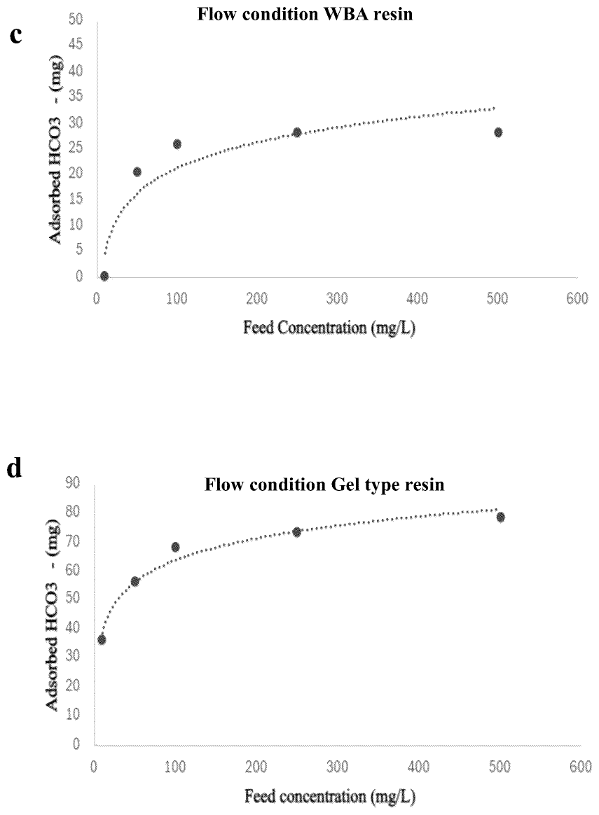

After saturation, the adsorbed bicarbonate ions were eluted from the resins using 100 ml of 1M NaCl. The eluted samples were collected for analysis. The amount of HCO₃⁻ adsorbed by the resins was quantified by titrating the eluted samples against a 0.001 M H₂SO₄ solution. The endpoint of titration was recorded to calculate the bicarbonate concentration. The quantity of bicarbonate ions adsorbed by the resins was calculated for each feed concentration. These values were plotted to create a graph (FIG. 5) showing the relationship between the bicarbonate feed concentration and the amount of bicarbonate ions (in mg) adsorbed by the resin. This graphical representation shows how different feed concentrations influence the adsorption capacity of the different resins tested providing insight into their performance under various contaminant loads.

FIG. 5(a) shows the calibration curve obtained for strong base type 1 anion exchange resin, FIG. 5(b) shows the calibration curve obtained for strong base type 2 anion exchange resin, FIG. 5(c) shows the calibration curve obtained for weak-base anion exchange resin, and FIG. 5(d) shows the calibration curve obtained for gel-type anion exchange resin.

The assessment of the strong base type 1 anion exchange resin, strong base type 2 anion exchange resin, weak-base anion exchange resin and gel-type anion exchange resin for bicarbonate binding under flow and no-flow conditions showed that among these resins, strong base type 1 and gel-type anion exchange resin exhibited the highest adsorption capacity under both flow and non-flow conditions. Based on this evaluation, the gel-type anion exchange resin and strong base type 1 resin were selected for subsequent field studies.

### Example 2: Field Experiments

Devices were assembled for field studies. Two types of devices were used—devices comprising a strong base type 1 anion exchange resin and devices comprising a gel-type anion exchange resin. Each device comprised about 6 g of resin and about 32 g of inert glass beads, corresponding to a resin-to-glass bead ratio of approximately 1:5 to 1:6. The strong base Type I resin used was either in the OH-form or the Cl⁻ form and was operational over a pH range of 0-14. The gel-type anion exchange resin used was in the Cl⁻ form and was also stable over a pH range of 0-14. The rigid, inert, non-porous material used was glass beads which provide structural stability and regulate pore connectivity of the medium. The size of the glass bead used was approximately 0.8 mm. This mixture of anion exchange resin and glass beads was used in the devices which had a cylindrical PVC housing (PVC pipe) and a nylon mesh for separating the intermediate column housing the anion exchange resin and glass bead mixture from the first chamber and the second chamber.

The devices were assembled as follows. The devices were constructed using a cylindrical housing from a 1-inch PVC pipe of approximately 6 cm length. Nylon mesh (200 mesh size, 10×10 cm) was secured at a distance from the downstream opening of the PVC pipe. The reactive medium comprising approximately 6 g of anion exchange resin uniformly mixed with 32 g of inert, glass beads, were introduced into the cartridge. Another nylon mesh (200 mesh size, 10×10 cm) was secured at a distance from the upstream opening of the PVC pipe to pack the reactive medium comprising anion exchange resin and beads. In other words, the nylon mesh was used at both the top and bottom of the resin-bead mixture to retain the mixture while allowing water to flow through the device. A smaller fitting tube with an external diameter of less than 1 inch (approximately 3 cm length) was attached to both ends of the device to secure the internal components.

Then the devices comprising the strong base type 1 anion exchange resin or gel-type anion exchange resin were buried in the ground for control and experimental runs:
1. In the control runs, the devices were buried in fields which were not treated for enhanced rock weathering, that is fields in which no external silicate substrate was added for enhanced rock weathering.
2. In the experimental runs, the devices were buried in fields which were treated for enhanced rock weathering, that is fields in which an external silicate substrate, namely, basalt, was added for enhanced rock weathering.

Devices of identical composition and design were subjected to experimental and control runs to ensure comparability of results.

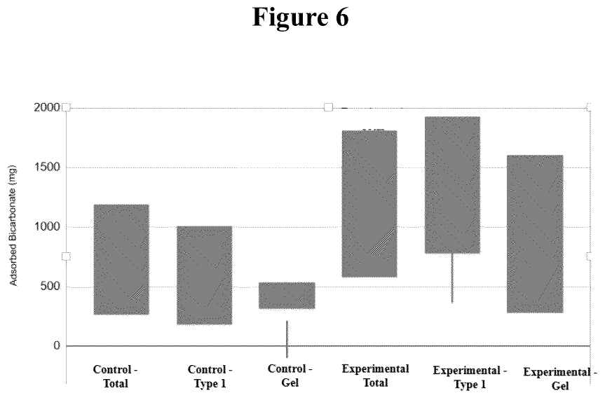

The deployed devices were removed after 120 days and the resins retrieved from the devices underwent laboratory elution and titration to quantify the captured bicarbonate ions and basic cations, which serve as direct proxies for sequestered carbon. The results in table 1 show the amount of bicarbonate ions (mg) retrieved in some of the control and experimental runs. FIG. 6 shows the average bicarbonate amount obtained from buried devices after elution. The results show that devices deployed under experimental conditions consistently resulted in higher bicarbonate recovery after elution compared to the control devices across all resin types.

TABLE 1
Field deployment results of control (non-basalt) and experimental
(basalt) fields. (T denotes type 1 resin; G denotes Gel resin; C
denotes control field; and D denotes deployed or experimental field)

| | | | HCO₃⁻ | HCO₃⁻ |
|---|---|---|---|---|
| | | | Concen- | mass |
| | Experiment | Name of | tration | obtained |
| Resin Type | Type | experiment | (mg/L) | (mg) |
| Strong base | Control | T1-Control | 690.52 | 55.2416 |
| type 1 | | | | |
| Strong base | Control | T2-Control | 980.88 | 78.4704 |
| type 1 | | | | |
| Strong base | Control | T3-Control | 612.44 | 48.9952 |
| type 1 | | | | |
| Strong base | Control | T5-Control | 961.36 | 76.9088 |
| type 1 | | | | |
| Gel-type | Control | G1-Control | 695.4 | 55.632 |
| Gel-type | Control | G2-Control | 822.28 | 65.7824 |
| Gel-type | Control | G3-Control | 1356.64 | 108.5312 |
| Gel-type | Control | G5-Control | 1224.88 | 97.9904 |
| Strong base | Experimental | T1- | 917.44 | 73.3952 |
| type 1 | | Experimental | | |
| Strong base | Experimental | T2- | 1227.32 | 98.1856 |
| type 1 | | Experimental | | |
| Strong base | Experimental | T3- | 2410.72 | 192.8576 |
| type 1 | | Experimental | | |
| Strong base | Experimental | T4- | 827.16 | 66.1728 |
| type 1 | | Experimental | | |
| Strong base | Experimental | T5- | 322.08 | 25.7664 |
| type 1 | | Experimental | | |
| Gel-type | Experimental | G2- | 907.68 | 72.6144 |
| | | Experimental | | |
| Gel-type | Experimental | G3- | 1551.84 | 124.1472 |
| | | Experimental | | |
| Gel-type | Experimental | G5- | 1329.8 | 106.384 |
| | | Experimental | | |
| Gel-type | Experimental | G6- | 1459.12 | 116.7296 |
| | | Experimental | | |

Overall, the present invention is a technical improvement to the process of MRV for ERW by utilizing anion-exchange resin for directly capturing bicarbonate ions throughout the given time. Specifically, the present invention reduces cost and increases efficiency by introducing a novel device and a unique methodology. These improvements allow for time-integrated, in situ quantification of carbon sequestration, which is essential for the high-accuracy requirements of the upcoming carbon credit market.

The terms and expressions employed herein are used as terms and expressions of description and not of limitation, and there is no intention, in the use of such terms and expressions, of excluding any equivalents of the features shown and described or portions thereof. In addition, having described certain embodiments of the invention, it will be apparent to those of ordinary skill in the art that other embodiments incorporating the concepts disclosed herein may be used without departing from the spirit and scope of the invention. The features and functions of the various embodiments may be arranged in various combinations and permutations, and all are considered to be within the scope of the disclosed invention. Accordingly, the described embodiments are to be considered in all respects as only illustrative and not restrictive. Furthermore, the configurations, materials, and dimensions described herein are intended as illustrative and in no way limiting. Similarly, although physical explanations have been provided for explanatory purposes, there is no intent to be bound by any particular theory or mechanism, or to limit the claims in accordance therewith.

### REFERENCES

Almaraz, Maya, Nina L. Bingham, Iris O. Holzer, Emily K. Geoghegan, Heath Goertzen, Jaeeun Sohng, and Benjamin Z. Houlton. “Methods for determining the CO2 removal capacity of enhanced weathering in agronomic settings.” Frontiers in Climate 4 (2022): 970429.
Amann, Thorben, and Jens Hartmann. “Carbon accounting for enhanced weathering.” Frontiers in Climate 4 (2022): 849948.
Kantola, Ilsa B., Elena Blanc-Betes, Michael D. Masters, Elliot Chang, Alison Marklein, Caitlin E. Moore, Adam von Haden et al. “Improved net carbon budgets in the US Midwest through direct measured impacts of enhanced weathering.” Global change biology 29, no. 24 (2023): 7012-7028.
Reershemius, Tom, Mike E. Kelland, Jacob S. Jordan, Isabelle R. Davis, Rocco D'Ascanio, Boriana Kalderon-Asael, Dan Asael et al. “Initial validation of a soil-based mass-balance approach for empirical monitoring of enhanced rock weathering rates.” Environmental Science & Technology 57, no. 48 (2023): 19497-19507.
Rebecca B. Neumann, Matthew L. Polizzotto, A. Borhan M. Badruzzaman, M. Ashraf Ali, Zhongyuan Zhang, Charles F. Harvey, “Hydrology of a groundwater-irrigated rice field in Bangladesh: Seasonal and daily mechanisms of infiltration”, Water Resources Research, Volume 45, Issue 9, September 2009
M. C. S. Wopereis, B. A. M. Bouman, M. J. Kropff, H. F. M. ten Berge, A. R. Maligaya, “Water use efficiency of flooded rice fields I. Validation of the soil-water balance model SAWAH” Agricultural Water Management, Volume 26, Issue 4, 1994, Pages 277-289
T. P. Tuong, M. C. S. Wopereis, J. A. Marquez, M. J. Kropff, “Mechanisms and Control of Percolation Losses in Irrigated Puddled Rice Fields”, Soil Science Society of America Journal, Volume 58, Issue 6, November-December 1994, Pages 1794-1803

We claim:

1. A medium for adsorbing and storing bicarbonate ions from a sample, wherein the medium comprises:
a. an anion exchange resin; and
b. a rigid, inert, non-porous material
wherein, the mass ratio of the anion exchange resin to the rigid, inert, non-porous material is from about 1:5 to 1:10, and wherein the sample is soil.

2. The medium of claim 1, wherein the mass ratio of the anion exchange resin to the rigid, inert, non-porous material is from about 1:5 to 1:6.

3. The medium of claim 1, wherein the anion exchange resin is selected from the group consisting of a strong-base anion exchange resin, a weak-base anion exchange resin, a gel-type anion exchange resin and combinations thereof.

4. The medium of claim 1, wherein the anion exchange resin is polystyrene-based anion exchange resin or acrylic polymer-based anion exchange resin.

5. The medium of claim 1, wherein the anion exchange resin is in hydroxide (OH⁻) form or chloride (Cl⁻) form or in both hydroxide (OH⁻) and chloride (Cl⁻) forms.

6. The medium of claim 1, wherein the anion exchange resin is operational over a pH range of 0 to 14.

7. The medium of claim 1, wherein the rigid, inert, non-porous material is selected from the group consisting of glass, quartz, ceramic and combinations thereof and is in the form of beads or particles.

8. The medium of claim 1, wherein the rigid, inert, non-porous material is glass.

9. The medium of claim 1, wherein the rigid, inert, non-porous material is in the form of beads and the beads have an average diameter between about 0.6 mm and 1 mm.

10. A device (1) for monitoring, reporting and verification of carbon dioxide removal in enhanced rock weathering, the device (1) comprising three chambers (2, 3, 4), the chambers being a first chamber (2), an intermediate chamber (3) and a second chamber (4), wherein the intermediate chamber (3) is present between the first chamber (2) and the second chamber (4)
the first chamber (2) and the second chamber (4) house soil and the intermediate chamber (3) houses a medium (7) for adsorbing and storing bicarbonate ions,
the first chamber (2) and second chamber (4) are separated from the intermediate chamber (3) by porous layers (5, 6), and
the porous layers (5, 6) are configured to prevent the movement of soil from the first chamber (2) and second chamber (4) into the intermediate chamber (3) while allowing the soil infiltrate to pass through the chambers (2, 3, 4) of device (1), and
wherein
the medium (7) for adsorbing and storing bicarbonate ions comprises an anion exchange resin and beads of a rigid, inert, non-porous material in a pre-determined mass ratio.

11. The device (1) of claim 10, wherein the pre-determined mass ratio of the anion exchange resin and rigid, inert, non-porous material is from about 1:5 to 1:10.

12. The device (1) of claim 11, wherein the pre-determined mass ratio of the anion exchange resin and rigid, inert, non-porous material is from about 1:5 to 1:6.

13. The device (1) of claim 10, wherein the rigid, inert, non-porous material is in the form of beads or particles.

14. The device (1) of claim 10, wherein the rigid, inert, non-porous material is in the form of beads and the beads have an average diameter between about 0.6 mm and 1 mm.

15. The device (1) of claim 10, wherein the rigid, inert, non-porous material is selected from the group consisting of glass, quartz, ceramic and combinations thereof.

16. The device (1) of claim 10, wherein the rigid, inert, non-porous material is glass.

17. The device (1) of claim 10, wherein the first and second chambers (2, 4):
a. get filled with soil when the device (1) is deployed in the field; or
b. are filled with soil prior to deploying device (1) in the field.

18. The device (1) of claim 10, wherein the anion exchange resin is selected from the group consisting of a strong-base anion exchange resin, a weak-base anion exchange resin, a gel-type anion exchange resin and combinations thereof.

19. The device (1) of claim 10, wherein the anion exchange resin is polystyrene-based anion exchange resin or acrylic polymer-based anion exchange resin.

20. The device (1) of claim 10, wherein the anion exchange resin is in hydroxide (OH⁻) form or chloride (Cl⁻) form or in both hydroxide (OH⁻) and chloride (Cl⁻) forms.

21. The device (1) of claim 10, wherein the anion exchange resin is operational over a pH range of 0 to 14.

22. The device (1) of claim 10, wherein the porous layers are a mesh or a porous substrate.

23. The device (1) of claim 10, wherein the porous layers are a nylon mesh, a filtration cloth, a microporous synthetic mesh, or combinations thereof.

24. The device (1) of claim 10, wherein the device comprises housing (8) which is composed of a material that is impermeable, water-resistant and capable of maintaining structural integrity and is selected from the group consisting of polyvinyl chloride, polypropylene, high density polyethylene, and is preferably polyvinyl chloride.

25. The device (1) of claim 10, wherein the device is designed to comply with equation (1)
K_avg=L₁/{(L₂/K₂)+(L₃/K₃)+(L₄/K₄)} (1)
wherein,
K_avg is the average permeability of the device in m/s;
L₁ is the total length of the device (1),
L₂, L₃ and L₄ are the lengths of the first (2), intermediate (3) and second (4) chambers, respectively, and
K₂, K₃ and K₄ are the permeability in m/s of the first (2), intermediate (3) and second (4) chambers, respectively.

26. The device of claim 10 which can be a preassembled device or is assembled in situ.

27. A method for monitoring, reporting and verification of carbon dioxide removal in enhanced rock weathering, comprising:
a. positioning one or more device (1) of claim 10 in soil such that an upstream opening (9) of the device (1) is arranged underneath a soil surface (11) in a field,
b. allowing water present in the field to pass through the device (1);
c. retrieving the device (1) from the soil after a pre-determined period of time; and
d. quantifying the bicarbonate ions taken up by the medium (7) for adsorbing and storing bicarbonate ions.

28. The method of claim 27, wherein the device (1) is either pre-assembled or is assembled in situ.

29. The method of claim 27, wherein the pre-determined period of time is at least one cropping season.

30. The method of claim 27, wherein the quantification of bicarbonate ions is carried out by elution and titration.

31. The method of claim 27, wherein the device (1) is positioned vertically in the soil.

32. The method of claim 27, wherein the device (1) is positioned near the bund in a field.

33. The method of claim 27, wherein multiple devices (1) are positioned near the bund in a field at a repeated and regular interval.

34. The method of claim 33, wherein multiple devices (1) are positioned along the bund in a field at a repeated and regular interval of 2 meters from each other.

35. Use of a device (1) of claim 10 for monitoring, reporting and verification of carbon dioxide removal in enhanced rock weathering.

36. Use of a method of claim 27, for monitoring, reporting and verification of carbon dioxide removal in enhanced rock weathering.
